(19) United States
(12) Patent Application Publication
(10) Pub. No.: US 2012/0220812 A1
(43) Pub. Date: Aug. 30, 2012

(54) ULTRASOUND NEUROMODULATION FOR STROKE MITIGATION AND REHABILITATION

(76) Inventor: David J. Mishelevich, Playa del Rey, CA (US)

(21) Appl. No.: 13/405,337

(22) Filed: Feb. 26, 2012

Related U.S. Application Data
(60) Provisional application No. 61/447,081, filed on Feb. 27, 2011.

(51) Int. Cl. A61N 7/00; A61N 5/00; A61N 2/00

(52) U.S. Cl. 600/2; 601/2; 600/9; 600/1

(57) ABSTRACT

Disclosed are methods and systems and methods for neuromodulation of the Motor Cortex and other areas of the brain impacted by stroke. The neuromodulation can produce acute effects or result in Long-Term Potentiation (LTP) or Long-Term Depression (LTD). Included is control of direction of the energy emission, intensity, pulse duration, frequency, firing pattern, and phase/intensity relationships to targeting and accomplishing up-regulation and/or down-regulation. The invention can be applied for mitigating the effects of stroke and/or in stroke rehabilitation.

### CROSS REFERENCE TO RELATED APPLICATIONS

This patent application claims priority to Provisional Patent Application No. 61/447,081, filed Feb. 27, 2011, entitled “ULTRASOUND NEUROMODULATION FOR STROKE MITIGATION AND REHABILITATION.” The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for ultrasound neuromodulation including one or more ultrasound sources for neuromodulation of target deep brain regions to up regulate or down regulate neural activity for the mitigation of the effects of and/or the rehabilitation of stroke.

### BACKGROUND OF THE INVENTION

It has been demonstrated that focused ultrasound directed at neural structures can stimulate those structures. If neural activity is increased or excited, the neural structure is up regulated; if neural activated is decreased or inhibited, the neural structure is down regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22 (2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3 (10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested. The above approach is incorporated in a patent application submitted by Tyler (Tyler, Tyler, William, James P., PCT/US2009/050560, WO 2010/009141, published 2011 Jan. 21).

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to produce a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 300 Hz.) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider (U.S. patent application Ser. No. 12/263,026 published as U.S. 2009/0112133 A1, Apr. 30, 2009) described an alternative approach in which neural transmission patterns between neural structures and/or regions are modified using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24 (2):275-83 and Clement G T, Hynynen K (2002) A non-invasive method for focusing ultrasound through the human skull. Phys Med Biol 47: 1219-1236.). Ultrasound can be focused to 0.5 to 2 mm as TMS to 1 cm at best.

### SUMMARY OF THE INVENTION

It is the purpose of this invention to provide methods and systems for non-invasive neuromodulation of selected portions of the brain to mitigate against the impacts of stroke and foster stroke rehabilitation. The latter can include Long-Term Potentiation (LTP). Included is control of direction of the energy emission, intensity, pulse duration, firing patterns, frequency, and phase/intensity relationships to targeting and accomplishing up-regulation and/or down-regulation. Use of ancillary monitoring or imaging to provide feedback is optional. In embodiments where concurrent imaging is performed, the device of the invention is to be constructed of non-ferrous material.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target. For example, some targets, like the motor cortex, are elongated and will be more effectively served with an elongated ultrasound field at the target.

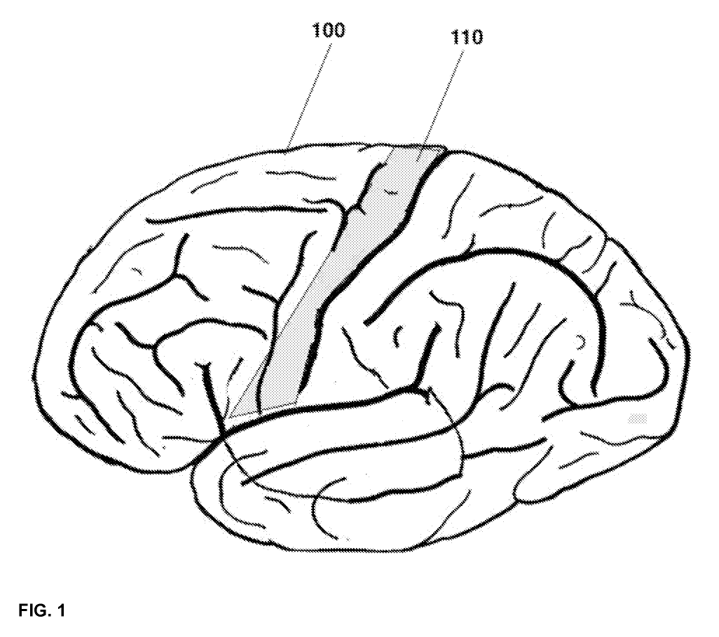

FIG. 1 shows a lateral view of the brain with the location of the motor cortex indicated.

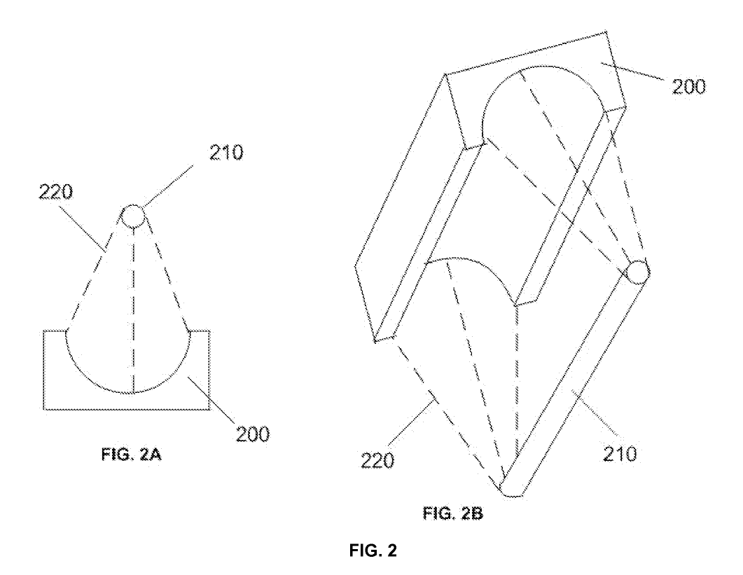

FIG. 2 illustrates two different view of an ultrasound transducer that produces an elongated beam to neuromodulate elongated targets.

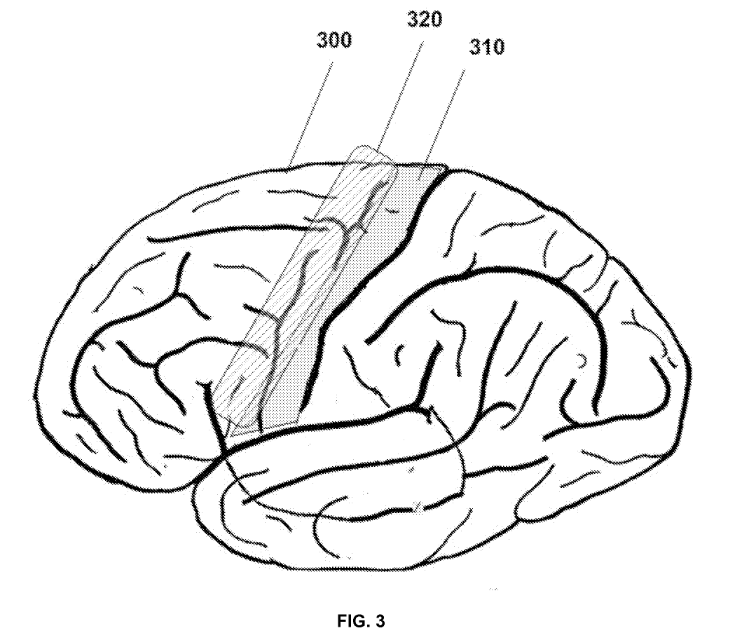

FIG. 3 demonstrates the positioning of an ultrasound transducer relative to the motor cortex.

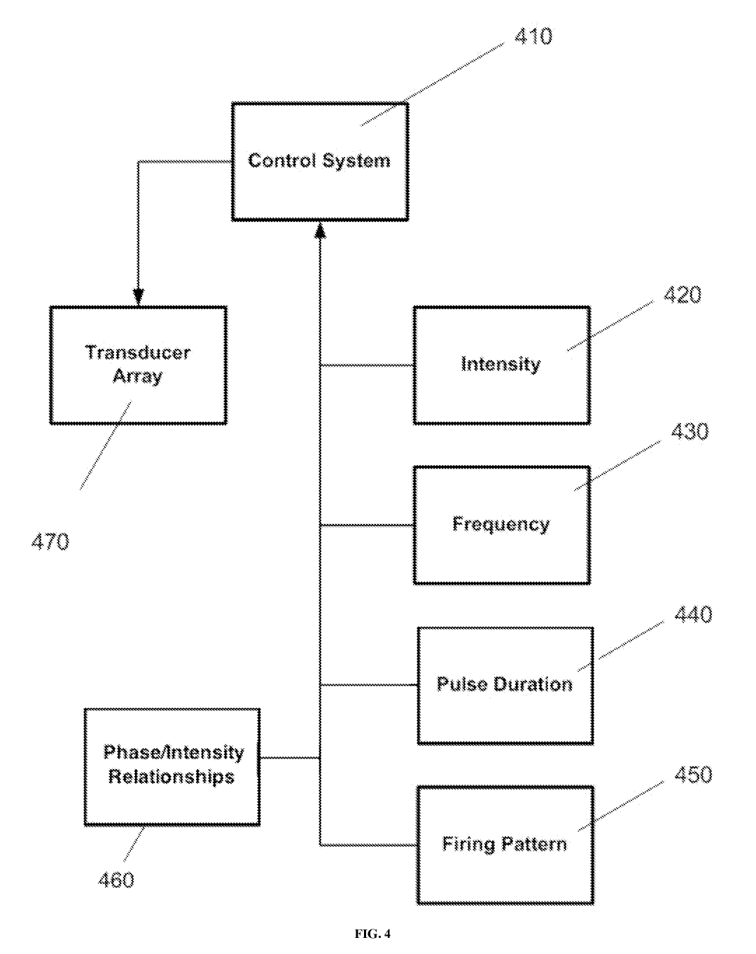

FIG. 4 shows a block diagram of the control circuit.

### DETAILED DESCRIPTION OF THE INVENTION

It is the purpose of this invention to provide methods and systems and methods for ultrasound neuromodulation of target deep brain regions to up-regulate or down-regulate neural activity for the mitigation of the effects of and/or the rehabilitation of stroke. This neuromodulation using ultrasound can produce acute effects or Long-Term Potentiation (LTP) or Long-Term Depression (LTD). Included is control of direction of the energy emission, intensity, pulse duration, frequency, firing patterns, and phase/intensity relationships.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz with power generally applied less than 60 mW/cm² but also at higher target- or patient-specific levels at which no tissue damage is caused. The acoustic frequency is gated at the lower rate to impact the neuronal structures as desired (e.g., say 300 Hz for inhibition (down-regulation) or 1 kHz for excitation (up-regulation). Ultrasound therapy can be combined with therapy using other devices (e.g., Transcranial Magnetic Stimulation (TMS)).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Blatek and Imasonic can also supply suitable ultrasound transducers. Other embodiments are applicable as well, including different transducer diameters, different frequencies, and different focal lengths. In an alternative embodiment, focus can be de-emphasized or eliminated with a smaller ultrasound transducer diameter with a shorter longitudinal dimension, if desired, as well. Ultrasound conduction medium will be required to fill the space.

FIG. 1 shows a brain 100 with cerebral folds with shaded area 110 denoting the location of the Primary Motor Cortex (designated as M1). The intervening bony skull is not shown. All or part of the motor cortex can be damaged by a stroke and other areas may be damaged by ischemic or hemorrhagic stroke as well. Typically the edges of an area impacted by a stroke are viable and neuromodulation of these edges can mitigate against further loss of tissue acutely. In the longer term, neuromodulation of this viable tissue can foster post-stroke rehabilitation. Besides Primary Motor Cortex, strokes cause lesions in the Primary Sensory Cortex, Wernicke's Area, posterior limb of internal capsule, basis pontis, corona radiate, and other neural centers.

FIG. 2 shows an ultrasound transducer array configured to produce an elongated pencil-shaped focused field. Such an array would he applied to stimulate an elongated target such as the motor cortex. Note that one embodiment is a swept-beam transducer with the capability of sweeping the sound field over any portion of the length of the ultrasound transducer. Thus it is possible to determine over what length of a target that the ultrasound is applied. For example, one could apply ultrasound to only the superior portion of the target. In FIG. 2A, an end view of the array is shown with curved-cross section ultrasonic array 200 forming a sound field 220 focused on target 210. FIG. 2B shows the same array in a side view, again with ultrasound array 200, target 210, and focused field 220. FIG. 3 shows for brain 300, the positioning of an ultrasound transducer 320 over Primary Motor Cortex 310. The intervening bony skull is not shown. The space between the surface of the ultrasound transducer and the surface of the head Is filled with ultrasound conduction medium (e.g., Dermasol from California Medical Innovations) (not shown) with a layer of ultrasound conduction gel between the surface of the ultrasound conduction medium and the surface of the head. One or more such ultrasound transducers may be aimed at other areas of the brain damaged by stroke. Stimulation can be unilateral or bilateral. It has been found using rTMS that there can be advantages to exciting the motor cortex ipsilateral to the brain lesion and inhibiting the motor cortex contralateral to the brain region.

Results with ultrasound neuromodulation would reflect what happens with Transcranial Magnetic Stimulation (TMS), but with the additional advantage of ultrasound neuromodulation being more focused. With respect to language, post-stroke aphasia has been successfully treated with 1 Hz rTMS to language sites (Barwood C H, Murdoch B E, Whelan B M, Lloyd D, Rick S, O'Sullivan J D, Coulthard A, Wong A., “Improved language performance subsequent to low-frequency rTMS in patients with chronic non-fluent aphasia post-stroke,” Eur J Neurol. 2010 Dec. 7. doi: 10.1111/j.1468-1331.2010.03284.x) resulting in improved naming performance, expressive language, and auditory comprehensive. Stimulation of Wernicke's area at 1 Hz in two patients with sensory dominant aphasia showed improvement when combined with language therapy (Kakuda W, Abo M, Uruma G, Kaito N, Watanabe M., “Low-frequency rTMS with language therapy over a 3-month period for sensory-dominant aphasia: case series of two post-stroke Japanese patients,” Brain Inj. 2010; 24 (9):1113-7).

Motor function has been successfully improved with high-frequency rTMS stimulation to the motor cortex in terms of motor recovery (Chang W H, Kim Y H, Bang O Y, Kim S T, Park Y H, Lee P K., “Long-term effects of rTMS on motor recovery in patients after subacute stroke,” J Rehabil Med. 2010 September; 42 (8):758-64), motor disability and dysphagia (Khedr E M, Fetoh N A., “Short- and long-term effect of rTMS on motor function recovery after ischemic stroke,” Restor Neurol Neurosci. 2010; 28 (4):545-59), upper limb function in combination with DCS stimulation (Nowak D A, Bösl K, Podubeckà J, Carey J R., “Noninvasive brain stimulation and motor recovery after stroke,” Restor Neurol Neurosci. 2010; 28 (4):531-44), upper limb with 5 Hz rTMS combined with extensor motor training, improved motor function and decreased spasticity with 1 Hz rTMS stimulation of the contralesional cerebral hemisphere followed by intense occupational therapy (Kakuda W, Abo M, Kobayashi K, Momosaki R, Yokoi A, Fukuda A, Ishikawa A, Ito H, Tominaga A., (Low-frequency repetitive transcranial magnetic stimulation and intensive occupational therapy for post-stroke patients with upper limb hemiparesis: preliminary study of a 15-day protocol,” Int J Rehabil Res. 2010 Jul. 6).

Ipsilateral primary motor cortical stimulation at 10 Hz in patients with problems with dexterity of the hand after stroke showed improvement of index finger and hand tapping in those with subcortical stroke; there was some deterioration in those with cortical stroke (Ameli M, Grefkes C, Kemper F, Riegg F P, Rehme A K, Karbe H, Fink G R, Nowak D A., “Differential effects of high-frequency repetitive transcranial magnetic stimulation over ipsilesional primary motor cortex in cortical and subcortical middle cerebral artery stroke,” Ann Neurol. 2009 September; 66 (3):298-309).

In patients with impaired upper-limb function, stimulation of the primary motor cortex with Theta Burst Stimulation (TBS) demonstrated increased excitability with intermittent TBS on the same side, but decreased excitability on the side with continuous TBS of the contralesional M1 (Ackerley S J, Stinear C M, Barber P A, Byblow W D., “Combining theta burst stimulation with training after subcortical stroke,” Stroke. 2010 July; 41 (7):1568-72. Epub 2010 May 20). Note that the lateral resulted in an overall decrease in upper-limb function.

In patients suffering from acute ischemic stroke, rTMS was done with one group treated at 1 Hz and the other 3 Hz; at 3 months, 1 Hz group demonstrated both decreased excitability of the non-stroke hemisphere and increased excitability of the stroke hemisphere while the 3 Hz group showed only increased excitability of the stroke hemisphere (Khedr E M, Abdel-Fadeil M R, Farghali A, Qaid M., “Role of 1 and 3 Hz repetitive transcranial magnetic stimulation on motor function recovery after acute ischaemic stroke,” Eur J Neurol. 2009 December; 16 (12):1323-30. Epub 2009 Sep. 23).

There is support for the concept of intrahemispheric balance with 5 Hz rTMS stimulation ipsilaterally or 1 Hz. Stimulation contralaterally (Emara T H, Moustafa R R, Elnahas N M, Elganzoury A M, Abdo T A, Mohamed S A, Eletribi M A., “Repetitive transcranial magnetic stimulation at 1Hz and 5Hz produces sustained improvement in motor function and disability after ischaemic stroke,” Eur J Neurol. 2010 September; 17 (9):1203-9. Epub 2010 Apr. 8).

rTMS stimulation at 3 Hz for the esophageal motor cortex in patients with dysphagia resulting from acute lateral medullary infarction (LMI) or other brainstem infarctions resulted in swallowing maintained over two months (Khedr E M and Abo-Elfetoh N., “Therapeutic role of rTMS on recovery of dysphagia in patients with lateral medullary syndrome and brainstem infarction,” J Neurol Neurosurg Psychiatry. 2010 May; 81 (5):495-9. Epub 2009 Oct. 14.).

Stroke patients may benefit from other interventions as well. For example, rTMS stimulation of the Left Dorsolateral Prefrontal cortex (DLPFC) if the affected side had a positive effect on mode in terms of decreased depression even though there was no impact on cognition, Kim et al. (Kim B R, Kim D Y, Chun M H, Yi J H, Kwon J S., “Effect of repetitive transcranial magnetic stimulation on cognition and mood in stroke patients: a double-blind, sham-controlled trial,” Am J Phys Med Rehabil. 2010 May; 89 (5):362-8).

Ultrasound transmission medium (e.g., Dermasol from California Medical Innovations or silicone oil in a containment pouch) is used as insert within the ultrasonic transducer. An layer of ultrasound conduction gel is placed between the face of the transducer or an associated lens and the surface of the head or the patient being imaged. The depth of the point where the ultrasound is focused depends on the shape of the transducer and setting of the phase and amplitude relationships of the elements of the ultrasound transducer array.

A distinct advantage of ultrasound neuromodulation is the small size of the device itself and low power requirements (for example, with respect to the apparatus required for Transcranial Magnetic Stimulation and power required to use it). Since ultrasound neuromodulation devices can easily be made portable, they can be practically used at home and convalescent facilities.

The location of the stroke is immaterial from the perspective of neuromodulation. It can be applied to strokes located in cortical, subcortical, brainstem, and other regions. The region impacted by stroke can be a single one such as a large infarct or multiple small ones. It also does not matter whether the stroke is ischemic and hemorrhagic. Not only does neuromodulation foster metabolic changes, the repetitive neuromodulation can retrain neural pathways to allow restore function.

Stimulation can be done unilaterally or bilaterally to see diagnostically which muscle or muscle groups are affected. Therapeutically, the ultrasound neuromodulation can be used to stimulate muscles to exercise them.

Another consideration is combination with neuromodulation of regions other than Motor Cortex. For example, neuromodulation of the Reticular Activating System to keep the general level of brain and base central activity up to prevent Central Nervous System failure.

Transducer array assemblies of the appropriate type may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon and Blatek in the U.S. are other custom-transducer suppliers. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

FIG. 4 shows an embodiment of a control circuit. The positioning and emission characteristics of transducer array 470 are controlled by control system 410 with control inputs for intensity 420, frequency 430, pulse duration 440, firing pattern 450, and phase/intensity relationships 460 for beam steering and focusing on neural targets.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

The invention can be applied for a variety of stroke-related clinical purposes such as reversibly putting a patient into a coma (for example for the purpose of protecting the brain of the patient after a stroke or head injury). Effects can be either acute or durable effect through Long-Term Potentiation (LTP) and/or Long-Term Depression (LTD). Since the effect is reversible putting the patient in even a vegetative state is safe if handled correctly. The application of LTP or LTD provides a mechanism for adjusting the bias of patient activity up or down. Appropriate radial (in-out) positions can be determined through patient-specific imaging (e.g., PET or fMRI) or set based on measurements to the mid-line. The positions can set manually or via a motor (not shown). The invention allows stimulation adjustments in variables such as, but not limited to, intensity, firing pattern, frequency, phase/intensity relationships, dynamic sweeps, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of deep-brain neuromodulation using ultrasound stimulation, the method comprising:
aiming one or a plurality of ultrasound transducer at the one or a plurality of stroke-related targets,
applying pulsed power to the ultrasound transducer via a control circuit whereby the results are selected from the group consisting of stroke mitigation and stroke rehabilitation.

2. The method of claim 1, further comprising aiming an ultrasound transducer neuromodulating the Primary Motor Cortex in a manner selected from the group of up-regulation, down-regulation.

3. The method of claim 1, wherein the acoustic ultrasound frequency is in the range of 0.3 MHz to 0.8 MHz.

4. The method of claim 1, where in the power applied is selected from the group selected from less than 60 mW/cm² and greater than 60 mW/cm² but less than that causing tissue damage.

5. The method of claim 1, wherein a stimulation frequency of less than 500 Hz or lower is applied for inhibition of neural activity.

6. The method of claim 10 wherein modulation frequency of lower than 500 Hz is divided into pulses 0.1 to 20 msec. repeated at frequencies of 2 Hz or lower for down regulation.

7. The method of claim 1, wherein the stimulation frequency for excitation is in the range of 500 Hz to 5 MHz.

8. The method of claim 12 wherein modulation frequency of 500 Hz or higher is divided into pulses 0.1 to 20 msec. repeated at frequencies higher than 2 Hz for up regulation.

9. The method of claim 1, wherein the focus area of the pulsed ultrasound is 0.5 to 150 mm in diameter.

10. The method of claim 1, wherein the stimulation is selected from the group of unilateral and bilateral.

11. The method of claim 1, wherein the one or a plurality of stroke-related targets is selected from the group consisting Primary Motor Cortex, Primary Sensory Cortex, Wernicke's Area, posterior limb of internal capsule, basis pontis, corona radiate, and other neural centers.

12. The method of claim 1, wherein the clinical function is selected from the group consisting of exciting the motor cortex ipsilateral to the brain lesion and inhibiting the motor cortex contralateral to the brain region.

13. The method of claim 1, wherein mechanical perturbations are applied radially or axially to move the ultrasound transducers.

14. The method of claim 1, wherein the device is portable such what it can be used at locations selected from the group consisting of home and convalescent facilities.

15. The method of claim 1, wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient actions.

16. The method of claim 1, wherein ultrasound therapy is combined with or replaced by one or more therapies selected from the group consisting of Transcranial Magnetic Stimulation (TMS), Deep-Brain Stimulation (DBS), application of optogenetics, radiosurgery, Radio-Frequency (RF) therapy, and medications.

17. The method of claim 1, wherein the location of the stroke is selected from the group consisting of cortical, subcortical, and brainstem.

18. The method of claim 1, wherein the cause of the stroke is selected from the group consisting of ischemic and hemorrhagic.

19. The method of claim 1 wherein the region type impacted by stroke is selected from the group consisting of single and multiple.

20. The method of claim 1 where neuromodulation for stroke is combined with the neuromodulation of the Reticular Activating System to keep the general level of brain and base central activity up to prevent Central Nervous System failure.
